(19) United States
(12) Patent Application Publication
(10) Pub. No.: US 2024/0118469 A1
(43) Pub. Date: Apr. 11, 2024

(54) FRONT LIGHT STERILIZATION MODULE AND DISPLAY DEVICE

(71) Applicant: Wistron Corporation, New Taipei City (TW)

(72) Inventor: Yi-Hau Shiau, New Taipei City (TW)

(73) Assignee: Wistron Corporation, New Taipei City (TW)

(21) Appl. No.: 18/089,578

(22) Filed: Dec. 28, 2022

(30) Foreign Application Priority Data
Oct. 11, 2022 (TW) 111138378

(51) Int. Cl. G02B 5/20; A61L 2/10; F21V 8/00

(52) U.S. Cl. CPC G02B 5/208; A61L 2/10; G02B 6/0065; G06F 3/0412

(57) ABSTRACT

A front light sterilization module and a display device including the front light sterilization module are provided. The front light sterilization module includes a light source, a light guide substrate, an ultraviolet light reflection layer, and a touch electrode layer. The light source includes a first light-emitting element for providing an illumination beam and a second light-emitting element for providing a sterilization beam. The light guide substrate is located on the transmission path of the illumination beam and the sterilization beam, wherein the light source is located at a side of the light guide substrate. The ultraviolet light reflection layer reflects the sterilization beam. The ultraviolet light reflection layer is located between the touch electrode layer and the light guide substrate.

### CROSS-REFERENCE TO RELATED APPLICATION

This application claims the priority benefit of Taiwan application serial no. 111138378, filed on Oct. 11, 2022. The entirety of the above-mentioned patent application is hereby incorporated by reference herein and made a part of this specification.

### BACKGROUND

### Technical Field

The disclosure relates to an optical module and an electronic device, an in particular relates to a front light sterilization module and a display device.

### Description of Related Art

In the current market of various consumer electronic products, various displays have been widely used in portable electronic devices as display screens, such as electronic paper displays used in e-books, or reflective displays.

In the prior art, an electronic paper display or a reflective display may use ambient light as incident light to irradiate the display layer for the purpose of display; therefore, no backlight is needed, which may save power consumption. In order to expand the range of application, a front light module is generally disposed above the front panel of the display device, and the light source is disposed to the side of the light guide panel (LGP), so that the light emitted by the light source may travel in a total internal reflection manner in the light guide panel. By disrupting the total internal reflection with the microstructure, the light is refracted toward the direction of the display, and the light source is converted into a surface light source with uniform optical distribution. The light refracted toward the display may then be reflected by the display and transmitted to the viewer. The purpose of disposing the front light module is to enable the display to provide sufficient incident light even in a place with insufficient ambient light for the user to view the display screen.

### SUMMARY

The disclosure provides a front light sterilization module and a display device, which has the advantage of thinning and a sterilization function.

A front light sterilization module of the disclosure includes a light source, a light guide substrate, an ultraviolet light reflection layer, and a touch electrode layer. The light source includes a first light-emitting element and a second light-emitting element. The first light-emitting element provides an illumination beam, and the second light-emitting element provides a sterilization beam. The light guide substrate is located on a transmission path of the illumination beam and the sterilization beam. The light source is located at a side of the light guide substrate. A material of the light guide substrate is an inorganic material. The light guide substrate has a first surface and a second surface opposite to each other, and the first surface of the light guide substrate is disposed with multiple optical microstructure. The ultraviolet light reflection layer reflects the sterilization beam, in which the second surface of the light guide substrate faces the ultraviolet light reflection layer. The ultraviolet light reflection layer is located between the touch electrode layer and the second surface of the light guide substrate.

A display device of the disclosure includes a front light sterilization module and a reflective display panel. The reflective display panel is located on a transmission path of an illumination beam, wherein the light guide substrate is located in front of the reflective display panel.

In an embodiment of the disclosure, the light guide substrate is a glass substrate.

In an embodiment of the disclosure, the ultraviolet light reflection layer is sputtered on the second surface of the light guide substrate.

In an embodiment of the disclosure, a thickness of the ultraviolet light reflection layer is 0.1 microns.

In an embodiment of the disclosure, a thickness of the light guide substrate is 100 microns to 400 microns.

In an embodiment of the disclosure, the touch electrode layer is sputtered on the ultraviolet light reflection layer.

In an embodiment of the disclosure, the illumination beam is a visible light beam, the sterilization beam is an ultraviolet light beam, and the light emission wavelength of the sterilization beam ranges between 200 nm and 280 nm.

In an embodiment of the disclosure, a thickness of the touch electrode layer is 80 microns.

In an embodiment of the disclosure, the display device further includes an optical adhesive layer located between the front light sterilization module and the reflective display panel, and the optical adhesive layer is an ultraviolet light beam cut-off layer.

In an embodiment of the disclosure, a thickness of the optical adhesive layer is 100 microns.

In an embodiment of the disclosure, the reflective display panel is an electronic paper display.

In an embodiment of the disclosure, the optical adhesive layer is located between the touch electrode layer and the reflective display panel.

Based on the above, the front light sterilization module and the display device of the disclosure may transmit the sterilization beam in the light guide substrate through the configuration of the ultraviolet light reflection layer to realize the sterilization function. In addition, through the configuration that the light guide substrate is formed of inorganic material, the ultraviolet light reflection layer may be directly sputtered on the surface of the light guide substrate, and the touch electrode layer may be directly formed on the ultraviolet light reflection layer, so that the front light sterilization module and the display device have the advantage of thinning.

In order to make the above-mentioned features and advantages of the disclosure comprehensible, embodiments accompanied with drawings are described in detail below.

### BRIEF DESCRIPTION OF THE DRAWING

FIG. 1 is a structural schematic diagram of a display device of an embodiment of the disclosure.

FIG. 2 is an exploded schematic diagram of the front light sterilization module of FIG. 1.

FIG. 3 is a schematic diagram of the transmittance curve of the ultraviolet light reflection layer of the front light sterilization module of FIG. 2 for different light emission wavelength bands.

FIG. 4 is a schematic diagram of the light path of the illumination beam of the front light sterilization module of FIG. 2.

FIG. 5 is a schematic diagram of the light path of the sterilization beam of the front light sterilization module of FIG. 2.

### DETAILED DESCRIPTION OF DISCLOSED EMBODIMENTS

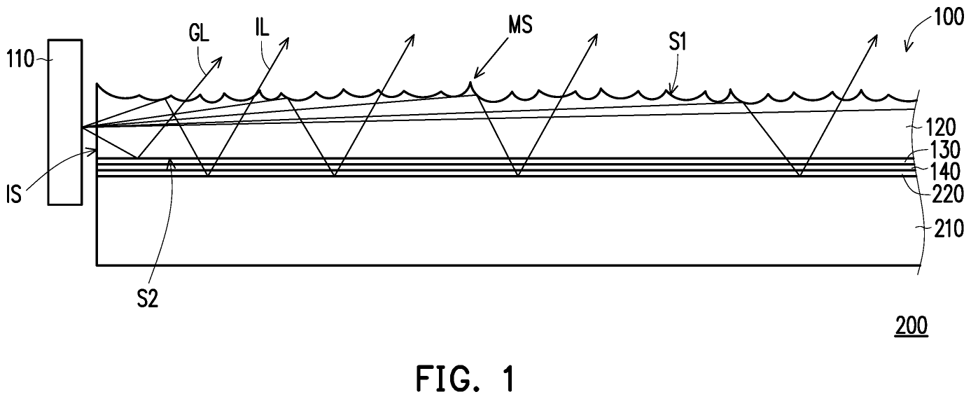

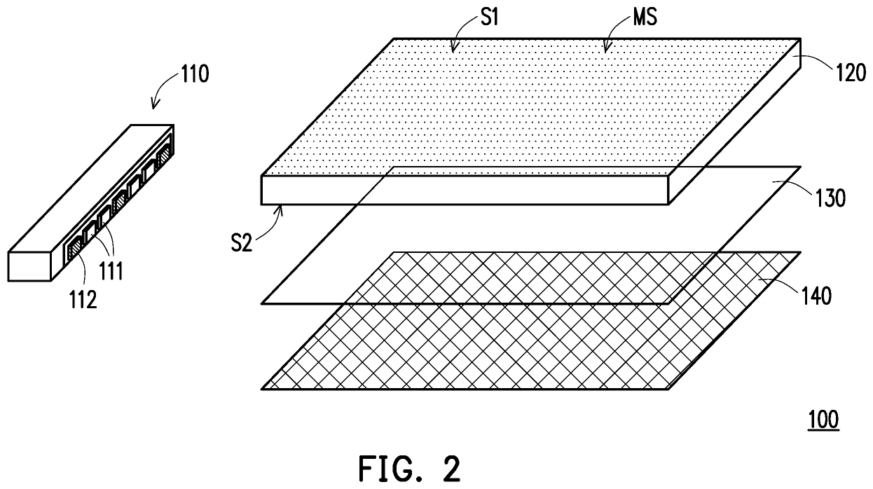

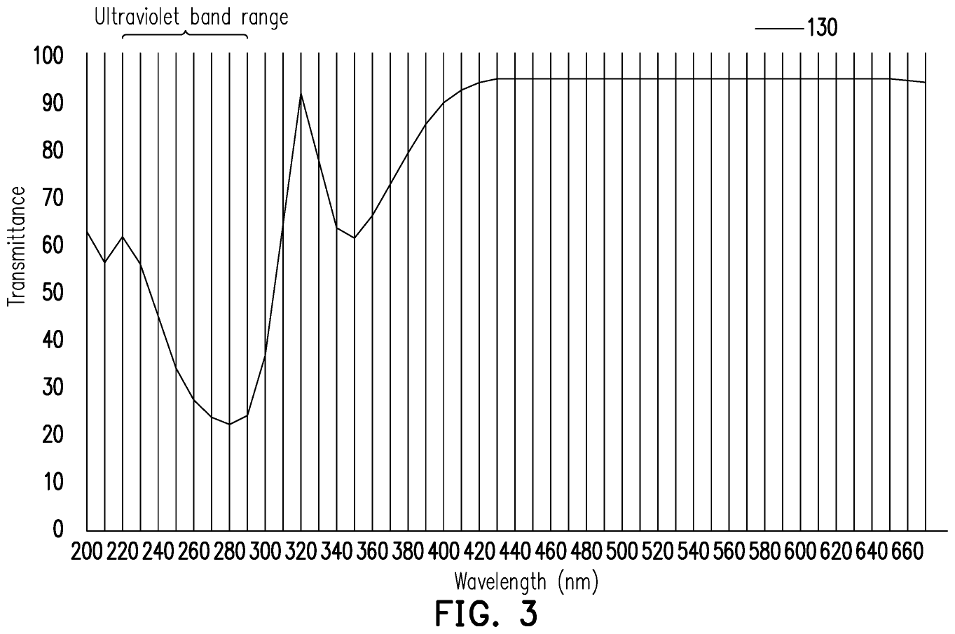

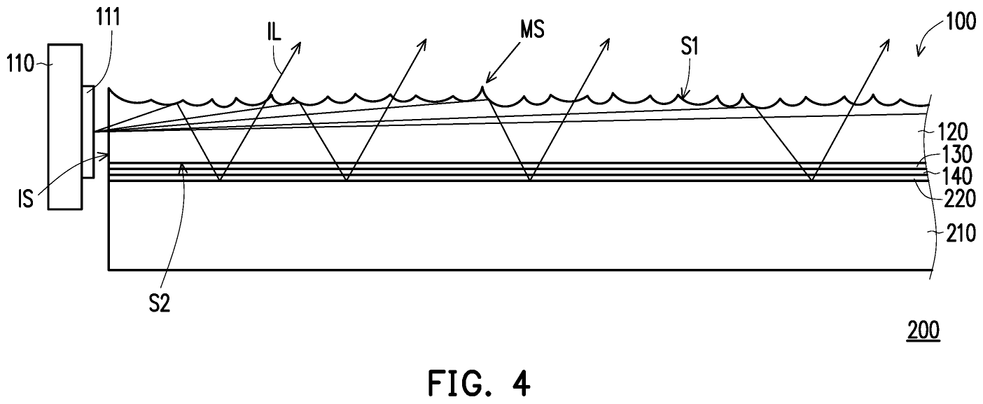

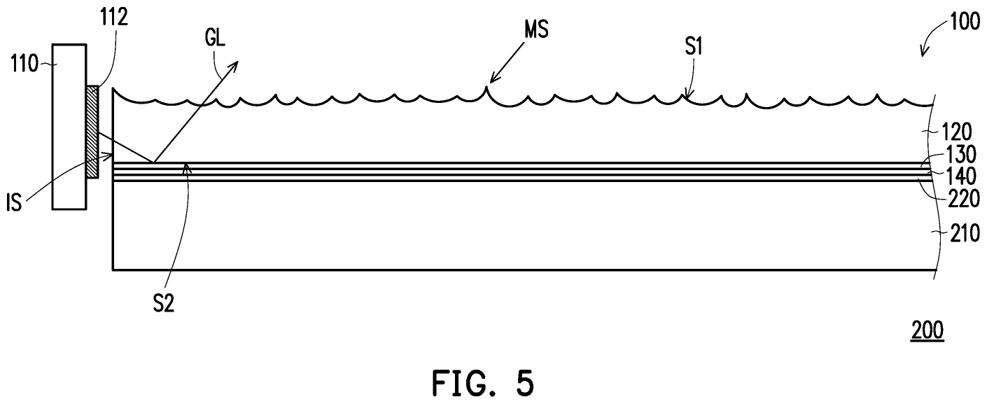

FIG. 1 is a structural schematic diagram of a display device of an embodiment of the disclosure FIG. 2 is an exploded schematic diagram of the front light sterilization module of FIG. 1. FIG. 3 is a schematic diagram of the transmittance curve of the ultraviolet light reflection layer of the front light sterilization module of FIG. 2 for different light emission wavelength bands. FIG. 4 is a schematic diagram of the light path of the illumination beam of the front light sterilization module of FIG. 2. FIG. 5 is a schematic diagram of the light path of the sterilization beam of the front light sterilization module of FIG. 2. Referring to FIG. 1 and FIG. 2, the display device 200 of this embodiment includes a front light sterilization module 100, an optical adhesive layer 220, and a reflective display panel 210. The optical adhesive layer 220 is located between the front light sterilization module 100 and the reflective display panel 210, so that the front light sterilization module 100 and the reflective display panel 210 are attached to each other. The front light sterilization module 100 includes a light source 110, a light guide substrate 120, an ultraviolet light reflection layer 130, and a touch electrode layer 140, wherein light source 110 is located at a side of the light guide substrate 120, the light guide substrate 120 is located in front of the reflective display panel 210, and the optical adhesive layer 220 is located between the touch electrode layer 140 and the reflective display panel 210. For example, in this embodiment, the reflective display panel 210 is an electronic paper display. In this way, in this embodiment, by adopting the reflective display panel 210, the generation of moiré may be suppressed.

Specifically, as shown in FIG. 2, FIG. 4 and FIG. 5, in this embodiment, the light source 110 includes at least one first light-emitting element 111 and at least one second light-emitting element 112. The first light-emitting elements 111 and the second light-emitting elements 112 are arranged in a staggered manner along the extending direction of the light source 110. At least one first light-emitting element 111 provides an illumination beam IL, and at least one second light-emitting element 112 provides a sterilization beam GL. For example, in this embodiment, the first light-emitting element 111 may adopt a light-emitting diode that emits a visible light beam, which may be provide the illumination beam IL, and the at least one second light-emitting element 112 may adopt a light-emitting diode that emits an ultraviolet light beam, which may be provide a sterilization beam GL, the light emission wavelength of the sterilization beam GL ranges, for example, between 200 nm and 280 nm.

Specifically, as shown in FIG. 2, the light guide substrate 120 has a first surface 51 and a second surface S2 opposite to each other, and the material of the light guide substrate 120 is an inorganic material. The optical microstructure MS and the ultraviolet light reflection layer 130 may be respectively fabricated on the first surface 51 and the second surface S2. For example, in this embodiment, the light guide substrate 120 may be a glass substrate, and multiple optical microstructures MS disposed on the first surface 51 of the light guide substrate 120 may adopt a glass etching process of the prior art. The glass etching process is, for example, to form a V-cut structure by pre-etching, and then perform further etching by a mixed acid etching process to form the desired optical structure.

In addition, by controlling the etching depth in the glass etching process, the roughness of the first surface 51 of the light guide substrate 120 may be adjusted, so that the user may have a good writing experience when writing on the first surface 51 with a stylus pen. For example, in this embodiment, the etching depth may be between 40 microns and 100 microns.

On the other hand, in this embodiment, the ultraviolet light reflection layer 130 may be formed on the second surface S2 of the light guide substrate 120 through sputtering, and, as shown in FIG. 3, in this embodiment, the ultraviolet light reflection layer 130 has a good capability to reflect ultraviolet light beams with light emission wavelengths ranging from 200 nm to 280 nm, and reflects the sterilization beam GL. Moreover, in this embodiment, the touch electrode layer 140 may also be fabricated directly on the ultraviolet light reflection layer 130 through sputtering. In this way, the front light sterilization module 100 as shown in FIG. 2 may be formed, in which the second surface S2 of the light guide substrate 120 faces the ultraviolet light reflection layer 130, and the ultraviolet light reflection layer 130 is located between the touch electrode layer 140 and the second surface S2 of the light guide substrate 120.

In this way, in the front light sterilization module 100, it is not necessary to additionally dispose a substrate for sputtering the touch electrode layer 140 as in the prior art, and the adhesive layer for attaching the substrate to which the touch electrode layer 140 is attached may also be omitted, so that a thinned size may be achieved. For example, in this embodiment, the thickness of the light guide substrate 120 is about 100 microns to 400 microns, the thickness of the ultraviolet light reflection layer 130 is only about 0.1 microns, the thickness of the touch electrode layer 140 is about 80 microns, and the thickness of the optical adhesive layer 220 is about 100 microns.

Further, as shown in FIG. 4 and FIG. 5, the light source 110 is located beside the light incident surface IS of the light guide substrate 120, and the light guide substrate 120 is located on the transmission path of the illumination beam IL and the sterilization beam GL. Furthermore, as shown in FIG. 4, when the light source 110 turns on the first light-emitting element 111, the illumination beam IL emitted by the light source 110 enters the light guide substrate 120 through the light incident surface IS of the light guide substrate 120, and travels in a total internal reflection manner in the light guide substrate 120. By disrupting the total internal reflection with the optical microstructure MS of the first surface 51 of the light guide substrate 120, the illumination beam IL is refracted toward the direction of the reflective display panel 210. Furthermore, as shown in FIG. 4, the reflective display panel 210 is also located on the transmission path of the illumination beam IL, and may reflect the light refracted toward the reflective display panel 210 such that the light is transmitted to the viewer. In this way, even in places with insufficient ambient light, sufficient incident light may still be provided for the user to view the display screen.

On the other hand, as shown in FIG. 5, when the light source 110 turns on the second light-emitting element 112 and the sterilization beam GL emitted by the light source 110 is refracted toward the direction of the reflective display panel 210, most of the sterilization beam GL is reflected back to the light guide substrate 120 by the ultraviolet light reflection layer 130 located between the light guide substrate 120 and the reflective display panel 210 and transmitted in the light guide substrate 120. In this way, the function of sterilizing the light guide substrate 120 may be realized, and the influence of the sterilization beam GL, which is an ultraviolet light beam, on the deterioration of the reflective display panel 210 may be suppressed. In addition, in this embodiment, the optical adhesive layer 220 may be an ultraviolet light beam cut-off layer, so that even if a small portion of the sterilization beam GL penetrates through the ultraviolet light reflection layer 130, it may be absorbed by the optical adhesive layer 220, thereby preventing the sterilization beam GL from deteriorating the reflective display panel 210.

To sum up, the front light sterilization module and the display device of the disclosure may transmit the sterilization beam in the light guide substrate through the configuration of the ultraviolet light reflection layer to realize the sterilization function. In addition, through the configuration that the light guide substrate is formed of inorganic material, the ultraviolet light reflection layer may be directly sputtered on the surface of the light guide substrate, and the touch electrode layer may be directly formed on the ultraviolet light reflection layer, so that the front light sterilization module and the display device have the advantage of thinning. In addition, by the configuration of the ultraviolet light reflection layer, the influence of the sterilization beam, which is an ultraviolet light beam, on the deterioration of the reflective display panel may also be suppressed. In addition, by controlling the depth dimension of the optical microstructure on the first surface of the light guide substrate, its roughness may also be adjusted, so that the user may have a good writing experience when writing with the stylus pen.

Although the disclosure has been described in detail with reference to the above embodiments, they are not intended to limit the disclosure. Those skilled in the art should understand that it is possible to make changes and modifications without departing from the spirit and scope of the disclosure. Therefore, the protection scope of the disclosure shall be defined by the following claims.

What is claimed is:

1. A front light sterilization module, comprising:
a light source, comprising a first light-emitting element and a second light-emitting element, wherein the first light-emitting element provides an illumination beam, and the second light-emitting element provides a sterilization beam;
a light guide substrate, located on a transmission path of the illumination beam and the sterilization beam, wherein the light source is located at a side of the light guide substrate, a material of the light guide substrate is an inorganic material, the light guide substrate has a first surface and a second surface opposite to each other, and the first surface of the light guide substrate is disposed with a plurality of optical microstructures;
an ultraviolet light reflection layer, wherein the ultraviolet light reflection layer reflects the sterilization beam, wherein the second surface of the light guide substrate faces the ultraviolet light reflection layer; and
a touch electrode layer, wherein the ultraviolet light reflection layer is located between the touch electrode layer and the second surface of the light guide substrate.

2. The front light sterilization module according to claim 1, wherein the light guide substrate is a glass substrate.

3. The front light sterilization module according to claim 1, wherein the ultraviolet light reflection layer is sputtered on the second surface of the light guide substrate.

4. The front light sterilization module according to claim 1, wherein a thickness of the ultraviolet light reflection layer is 0.1 microns.

5. The front light sterilization module according to claim 1, wherein a thickness of the light guide substrate is 100 microns to 400 microns.

6. The front light sterilization module according to claim 1, wherein the touch electrode layer is sputtered on the ultraviolet light reflection layer.

7. The front light sterilization module according to claim 1, wherein the illumination beam is a visible light beam, the sterilization beam is an ultraviolet light beam, and the light emission wavelength of the sterilization beam ranges between 200 nm and 280 nm.

8. The front light sterilization module according to claim 1, wherein a thickness of the touch electrode layer is 80 microns.

9. A display device, comprising:
a front light sterilization module, comprising:
a light source, comprising a first light-emitting element and a second light-emitting element, wherein the first light-emitting element provides an illumination beam, and the second light-emitting element provides a sterilization beam;
a light guide substrate, located on a transmission path of the illumination beam and the sterilization beam, wherein the light source is located at a side of the light guide substrate, a material of the light guide substrate is an inorganic material, the light guide substrate has a first surface and a second surface opposite to each other, and the first surface of the light guide substrate is disposed with a plurality of optical microstructures;
an ultraviolet light reflection layer, wherein the ultraviolet light reflection layer reflects the sterilization beam, wherein the second surface of the light guide substrate faces the ultraviolet light reflection layer; and
a touch electrode layer, wherein the ultraviolet light reflection layer is located between the touch electrode layer and the second surface of the light guide substrate;
a reflective display panel, located on a transmission path of an illumination beam, wherein the light guide substrate is located in front of the reflective display panel.

10. The display device according to claim 9, wherein the light guide substrate is a glass substrate.

11. The display device according to claim 9, wherein the ultraviolet light reflection layer is sputtered on the second surface of the light guide substrate.

12. The display device according to claim 9, wherein the touch electrode layer is sputtered on the ultraviolet light reflection layer.

13. The display device according to claim 9, wherein the illumination beam is a visible light beam, the sterilization beam is an ultraviolet light beam, and the light emission wavelength of the sterilization beam ranges between 200 nm and 280 nm.

14. The display device according to claim 9, further comprising:
an optical adhesive layer, located between the front light sterilization module and the reflective display panel, wherein the optical adhesive layer is an ultraviolet light beam cut-off layer.

15. The display device according to claim 14, wherein a thickness of the optical adhesive layer is 100 microns.

16. The display device according to claim 9, wherein a thickness of the ultraviolet light reflection layer is 0.1 microns.

17. The display device according to claim 9, wherein a thickness of the light guide substrate is 100 microns to 400 microns.

18. The display device according to claim 9, wherein a thickness of the touch electrode layer is 80 microns.

19. The display device according to claim 9, wherein the reflective display panel is an electronic paper display.

20. The display device according to claim 14, wherein the optical adhesive layer is located between the touch electrode layer and the reflective display panel.
